(19) United States
(12) Patent Application Publication
(10) Pub. No.: US 2019/0038869 A1
(43) Pub. Date: Feb. 7, 2019

(54) NEEDLE CATHETER FOR SUPPLYING DRUG

(71) Applicant: THEINGMEDICAL CO., LTD., Okcheon-gun, Chungcheongbuk-do (KR)

(72) Inventors: Jae Hyun PARK, Pyeongtaek-si (KR); Hee Dai KIM, Daejeon (KR)

(73) Assignee: THEINGMEDICAL CO., LTD., Okcheon-gun, Chungcheongbuk-do (KR)

(21) Appl. No.: 16/085,818

(22) PCT Filed: Mar. 20, 2017
(86) PCT No.: PCT/KR2017/002982

§ 371 Date: Sep. 17, 2018

(30) Foreign Application Priority Data
Mar. 18, 2016 (KR) 10-2016-0033031

(51) Int. Cl. A61M 25/00

(52) U.S. Cl. CPC A61M 25/0084; A61M 2205/3334; A61M 2025/009

(57) ABSTRACT

A needle catheter for injecting a drug is provided, the needle catheter includes a needle assembly for discharging a drug, a drug supply tube for supplying the drug to the needle assembly, and a drug regulation handle for regulating an amount of the drug to be supplied through the drug supply tube, wherein the needle assembly includes a first needle communicatively coupled to the drug supply tube and a second needle coupled to the drug supply tube in parallel with the first needle, and the first needle and the second needle are contact-coupled to each other by a partition wall.

### TECHNICAL FIELD

The present invention relates to a needle assembly, and more particularly, to the needle assembly capable of rapidly injecting a drug into a body.

### BACKGROUND ART

To order to inhibit a bleeding of an esophagus and gastrointestinal tract, a needle catheter for injecting a drug may be used to inject a hardener into a lesion through an endoscope or to inject a drug such as a cell line when performing a polyp removal treatment through the endoscope.

FIG. 1 is a perspective view showing a configuration of a conventional needle catheter. As shown in FIG. 1, the conventional needle catheter 10 includes an injection needle 11, a supply tube 13 for supplying a drug to the injection needle 11, and a regulating handle 15 connected to a drug pack (not shown) to regulate an amount of the drug supplied from the drug pack (not shown) to the supply tube 13.

Herein, the conventional injection needle 11 may be formed in a tubular shape having a predetermined length as shown in an enlarged view, and the drug may be injected into a body through a discharging hole formed at one end of the injection needle.

When performing the removal treatment of a large polyp or a plurality of polyps, a large amount of drug such as a cell line may be injected. However, since the conventional injection needle 11 has a constant diameter r1 ranging from 0.2 to 0.3 mm, the amount of the drug injected into the body through the injection needle 11 may be limited.

If the injection of the drug is delayed, a hemostasis may be delayed. In addition, if the hemostasis is not properly achieved, a bacterial and gastric acid may cause an inflammation reaction and bacterial infection. Thus, if the inflammation persists, ulcers may develop and result in poor prognosis.

### DISCLOSURE

### Technical Problem

An object of the present invention is to solve the above-described problems and provide a needle catheter capable of promptly injecting a large amount of drug into a body.

Another object of the present invention is to provide a needle catheter capable of rapidly injecting a drug by diversifying injection paths of the drug.

The objects and various advantages of the present invention will become more apparent from the preferred embodiments of the present invention by those skilled in the art.

### Technical Solution

The object of the present invention may be achieved by a needle catheter which injects a drug into a body. The needle catheter of the present invention may include a needle assembly for discharging the drug, a drug supply tube for supplying the drug to the needle assembly, and a drug regulation handle for regulating the amount of the drug to be supplied through the drug supply tube, wherein the needle assembly may include a first needle communicatively coupled to the drug supply tube; and a second needle coupled to the drug supply tube in parallel with the first needle, and the first needle and the second needle may be contact-coupled to each other by a partition wall.

According to one embodiment, the first needle and the second needle may be formed to have a plurality of intermediate discharging holes for discharging the drug into the body on outer circumferential surfaces of the needles.

### Advantageous Effects

The needle catheter in accordance with the present invention may increase the amount of the drug injected into the body by discharging the drug into the body with two needles at the same time.

In addition, the plurality of discharging holes may be formed on the surfaces of the needles to diversify the paths for injecting the drug into the body and increase an amount of the drug supplied to the body.

Thus, the drug may be quickly injected into the body to conduct the hemostasis rapidly when performing the polyp removal treatment.

### DESCRIPTION OF DRAWINGS

FIG. 1 is a perspective view showing a configuration of a conventional needle catheter;

FIG. 2 is a perspective view showing a configuration of a needle catheter in accordance with the present invention; and

FIG. 3 is a view illustrating a process of injecting a drug into a body using a needle catheter in accordance with the present invention.

### EXPLANATION OF SYMBOLS

100: needle catheter

110: needle assembly

111: first needle

111a: first ended discharging hole

111b: first intermediate discharging hole

113: second needle

113a: second ended discharging hole

113b: second intermediate discharging hole

115: partition wall

120: drug supply tube

130: regulating handle

### MODE

In order to fully understand the present invention, a preferred embodiment of the present invention will be described with reference to the accompanying drawings. The embodiments of the present invention may be modified in various forms and the scope of the present invention should not be construed as being limited to the embodiments described in detail below. The present embodiments may be provided to enable those skilled in the art to more fully understand the present invention. Therefore, the shape and the like of the elements in the drawings may be exaggerated in order to emphasize a clearer explanation. It should be noted that in the drawings, the same members are denoted by the same reference numerals. In addition, the detailed descriptions of well-known functions and configurations that may unnecessarily obscure the gist of the present invention may be omitted.

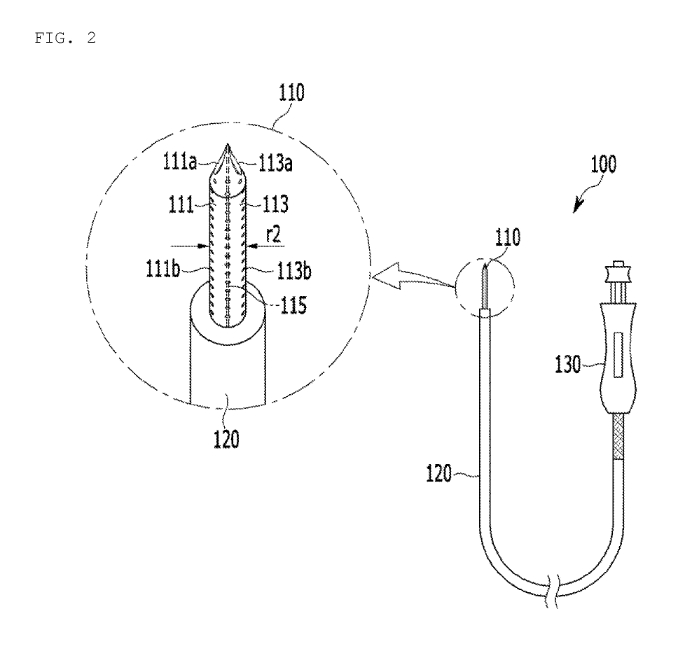

FIG. 2 is a perspective view showing a configuration of a needle catheter 100 for injecting a drug in accordance with the present invention. As shown in FIG. 2, the needle catheter 100 for injecting the drug may include a needle assembly 110 for injecting the drug into a body, a drug supply tube 120 for supplying the drug to the needle assembly 110, and a regulating handle 130 for regulating the amount of the drug supplied from a drug pack (not shown) or an aqueous solution pack (not shown) to the supply tube 120.

The needle assembly 110 in accordance with the present invention may be arranged such that a pair of needles 111 and 113 may be coupled to each other. That is, the first needle 111 and the second needle 113 may be formed in parallel to each other around a partition wall 115. The first needle 111 and the second needle 113 may be arranged to be communicated with the drug supply tube 120 to inject the drug A into the body at the same time.

The first needle 111 and the second needle 113 may be formed by sharply polishing end portions thereof at an angle of 30 to 60 degrees. Thus, the first needle 111 and the second needle 113 may be easily inserted into the body. The end portions of the first needle 111 and the second needle 113 may be provided with ended discharging holes 111a and 113a, respectively, such that the drug may be injected into the body.

Meanwhile, the first needle 111 and the second needle 113 may be formed to have a cross section of a semicircular shape, and may be arranged so as to share the partition wall 115 with each other. In this case, a plurality of intermediate discharging holes 111b and 113b may be formed on outer circumferential surfaces of the first needle 111 and the second needle 113 along the longitudinal direction of the needles.

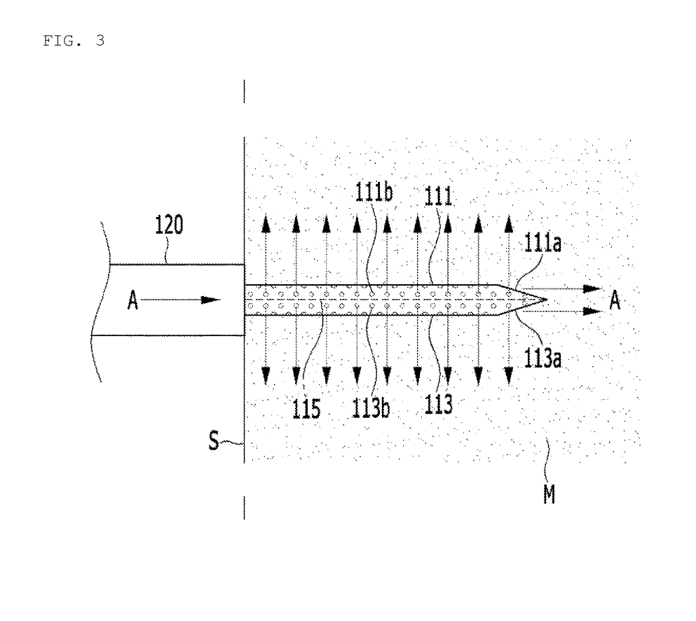

As shown in FIG. 3, when the drug A is supplied, the drug A may be moved along the first needle 111 and the second needle 113 and may be injected into the body along the plurality of intermediate discharging holes 111b and 113b. In addition, the drug A may be injected into the body through the ended discharging holes 111a and 113a as well.

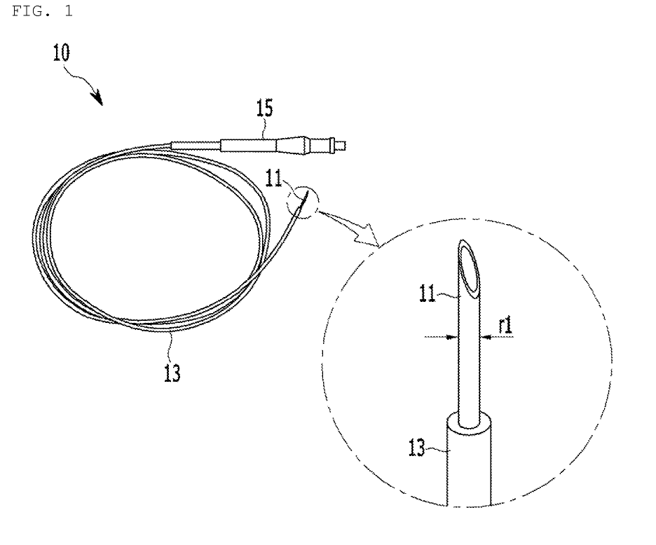

The conventional injection needle 11 shown in FIG. 1 has only one path for discharging the drug, so that the amount of the drug injected into the body may be limited.

Meanwhile, the needle assembly 110 in accordance with the present invention may be configured such that the drug A supplied through the drug supply tube 120 may be moved along the longitudinal direction of the first needle 111 and the second needle 113 and may be supplied to the body along the intermediate discharging holes 111b and 113b each formed on a middle of the first and second injection needles. In addition, the drug A may be supplied to the body through the ended discharging holes 111a and 113a as well.

Therefore, since a large amount of drug may be simultaneously injected into the body through various paths, there is an effect that an injection rate of the drug may be fast and the large amount of drug may be rapidly injected.

Herein, since a total diameter r2 of the first needle 111 and the second needle 113 may be a sum of the diameters of the first needle 111 and the second needle 113, the total diameter r2 may correspond to 1.5 to 2 times the diameter r1 of the conventional injection needle.

The diameters of the intermediate discharging holes 111b and 113b may be regulated differently depending on the type of a surgery or treatment in which the needle catheter 100 for injecting the drug is used and the type of the drug to be injected.

The drug supply tube 120 may be provided with the needle assembly 110 and the regulating handle 130. The drug supply tube 120 may be variously provided depending on the type of the drug to be supplied.

The regulating handle 130 may be designed with a convenient design so that a practitioner may easily regulate the amount of the drug to be supplied.

A drug injection process using the needle catheter 100 for injecting the drug in accordance with the present invention having such a configuration will be described with reference to FIGS. 2 and 3.

The needle catheter 100 for injecting the drug in accordance with the present invention may be used when performing a treatment for removing a polyp and the like which is found when examining an esophagus, stomach cancer, colon cancer and the like in a hospital.

A drug such as a cell line may be injected using the needle catheter 100 for injecting the drug in accordance with the present invention.

A sharp tip of the needle assembly 110 may be inserted into a treatment area of a patient. Then, the drug pack (not shown) may be connected to the regulating handle 130 and the amount of the drug to be injected into the body may be regulated.

The drug may be moved along the drug supply tube 120 and then supplied to the body through the needle assembly 110. In this case, as shown in FIG. 3, the drug A may be moved along the first needle 111 and the second needle 113 and supplied into the body through the plurality of intermediate discharging holes 111b and 113b formed on outer wall surfaces of the needles.

In addition, the drug A may be supplied into the body through the ended discharging holes 111a and 113a formed at tips of the first needle 111 and the second needle 113.

The needle assembly 110 in accordance with the present invention may discharge the drug into the body through the pair of needles 111 and 113 at the same time and inject the drug into the body using various paths through the plurality of intermediate discharging holes 111b and 113b formed on the surfaces of the needles 111 and 113.

Accordingly, the amount of the drug to be injected into the body may be significantly increased as compared to that of the drug to be injected by the conventional needle. Therefore, the large amount of the drug may be injected quickly to conduct the hemostasis rapidly.

As described above, the needle catheter for injecting the drug in accordance with the present invention may increase the amount of the drug to be injected into the body by discharging the drug into the body using two needles at the same time.

In addition, the plurality of discharging holes may be formed on the surfaces of the needles to diversify the paths for injecting the drug into the body and increase the amount of the drug to be supplied.

Accordingly, the drug may be quickly injected when performing the polyp removal treatment to conduct the hemostasis rapidly.

The embodiments of the needle catheter for injecting the drug in accordance with the present invention described above may be merely illustrative and it will be understood by those skilled in the art that various modifications and equivalent embodiments may be possible without departing from the scope of the present invention. Thus, it will be understood that the present invention may be not limited to the form set forth in the foregoing description. Accordingly, the true scope of the present invention should be determined by the technical idea of the appended claims. Also, it may be understood that the present invention includes all modifications, equivalents, and alternatives falling within the spirit and scope of the invention as defined by the appended claims.

1. A needle catheter for injecting a drug into a body, comprising:
a needle assembly for discharging the drug;
a drug supply tube for supplying the drug to the needle assembly; and
a drug regulation handle for regulating an amount of the drug to be supplied through the drug supply tube,
wherein the needle assembly includes a first needle communicatively coupled to the drug supply tube, and a second needle coupled to the drug supply tube in parallel with the first needle, and
wherein the first needle and the second needle are contact-coupled to each other by a partition wall and a plurality of intermediate discharging holes for discharging the drug into the body are formed on outer circumferential surfaces of the first and second needles to increase an injection rate of the drug.
